(19) United States
(12) Patent Application Publication
(10) Pub. No.: US 2005/0209670 A1
(43) Pub. Date: Sep. 22, 2005

(54) Stent delivery system with diameter adaptive restraint

(75) Inventors: William R. George, Santa Cruz, CA (US); Julian Nikolchev, Portola Valley, CA (US); Nicholas C. De Beer, Montara, CA (US); Dai T. Ton, Milpitas, CA (US); Frank Becking, Palo Alto, CA (US)

Correspondence Address: BOZICEVIC, FIELD & FRANCIS LLP (CARDIOMIND), 1900 UNIVERSITY AVENUE, SUITE 200, EAST PALO ALTO, CA, 94303, US

(73) Assignee: CardioMind, Inc.

(21) Appl. No.: 10/792,657

(22) Filed: Mar. 2, 2004

(51) Int. Cl. A61F 2/06

(52) U.S. Cl. 623/1.11

(57) ABSTRACT

Medical device and methods for delivery or implantation of prostheses within hollow body organs and vessels or other luminal anatomy are disclosed. The subject technologies may be used in the treatment of atherosclerosis in stenting procedures. For such purposes, a self-expanding stent may be deployed in connection with an angioplasty procedure with a stent delivery system having a diameter adaptive restraint. Upon withdrawal of the restraint, the stent is freed, while the restraint or connections thereto assumes a reduced diameter within a tubular body of the delivery guide.

### FIELD OF THE INVENTION

The present invention relates generally to medical device and methods. More particularly, it relates to delivery systems for implanting prostheses within hollow body organs and vessels or other luminal anatomy.

### BACKGROUND OF THE INVENTION

Implants such as stents and occlusive coils have been used in patients for a wide variety of reasons. One of the most common “stenting” procedures is carried out in connection with the treatment of atherosclerosis, a disease which result in a narrowing and stenosis of body lumens, such as the coronary arteries. At the site of the narrowing (i.e., the site of a lesion) a balloon is typically dilatated in an angioplasty procedure to open the vessel. A stent is set in apposition to the interior surface of the lumen in order to help maintain an open passageway. This result may be effected by means of scaffolding support alone or by virtue of the presence of one or more drugs carried by the stent aiding in the prevention of restenosis.

Various stent designs. have been developed and used clinically, but self-expandable and balloon-expandable stent systems and their related deployment techniques are now predominant. Examples of self-expandable stents currently in use are the Magic WALLSTENT® stents and Radius stents (Boston Scientific). A commonly used balloon-expandable stent is the Cypher® stent (Cordis Corporation). Additional self-expanding stent background is presented in: “An Overview of Superelastic Stent Design,” Min. Invas Ther & Allied Technol 2002: 9(3/4) 235-246, “A Survey of Stent Designs,” Min. Invas Ther & Allied Technol 2002: 11(4) 137-147, and “Coronary Artery Stents: Design and Biologic Considerations,” Cardiology Special Edition, 2003: 9(2) 9-14, “Clinical and Angiographic Efficacy of a Self-Expanding Stent” Am Heart J 2003: 145(5) 868-874.

Because self-expanding prosthetic devices need not be set over a balloon (as with balloon-expandable designs), self-expanding stent delivery systems can be designed to a relatively smaller outer diameter than their balloon-expandable counterparts. As such, self-expanding stents may be better suited to reach the smallest vasculature or achieve access in more difficult cases.

To realize such benefits, however, there continues to be a need in developing improved delivery systems. Problems encountered with known systems include drawbacks ranging from failure to provide means to enable precise placement of the subject prosthetic, to a lack of space efficiency in delivery system design. Poor placement hampers stent efficacy. Space inefficiency in system design prohibits scaling the systems to sizes as small as necessary to enable difficult access or small-vessel procedures (i.e., in tortuous vasculature or vessels having a diameter less than 3 mm, even less than 2 mm).

One known stent delivery system comprises a simple sheath set over a pusher in abutment with a stent. An example of such a system is disclosed in U.S. Pat. No. 4,580,568. Though elegant in design, the system fails to offer desired functional characteristics. Particularly, such a system is prone to misuse when a physician who in not intimately familiar with the hardware retracts or pushes the wrong one of the stent-abutting member or the sheath in an effort to free the stent. Dedicated handle systems have been developed to address this problem. Examples are provide in WO 99/04728, WO 00/18330, WO 98/23241, EP-A-747021, DE-A-44 20142 and U.S. Pat. No. 5,433,723.

Even when not misused, simple sheath system present issues with precise stent placement stemming from the fact that the sheath cannot be locked-down at the proximal end of an access catheter (e.g., at a hemostatic valve) while deploying the stent. As a result, it is difficult to prevent inadvertent axial movement of the stent. Because the sheath cannot be held onto, stent deployment requires that a user hold the pusher member (or handle attached thereto) steady while withdrawing the sheath in order to avoid pushing the stent forward within the vessel thereby complicating stent placement or producing “skid-marks” and even vessel perforation.

The system described in U.S. Pat. No. 5,534,007 assigned to SciMed Life Systems, Inc. offers an alternative to a simple-sheath type system for deploying self-expandable stents. The proximal end of the noted system can be locked-down, without the stent moving axially upon withdrawing its restraint. Yet, the system requires a collapsible, bellows-type sheath portioned between the stationary proximal sleeve and the moveable distal restraint. Furthermore, the system is deployed over a guidewire. Because of the large “over-the-guidewire size” and increasing size of the device resulting by compression of the bellows, the device is not able access or to be withdrawn from the smallest and/or most tortuous anatomy.

Accordingly, there exists a need for a system to better enable precise stent placement than a simple sheath system, but offering improved space efficiency over other know self-expanding stent delivery systems such as that in the '007 patent. Those with skill in the art may also appreciate further advantages or benefits of the invention.

### SUMMARY OF THE INVENTION

The present invention offers a stent delivery system in which a restraint holding a stent in position for deployment is adapted to collapse radially upon withdrawal from the stent. This diameter adaptive restraint enables the system to operate in a highly space efficient manner. Furthermore, it opens possibilities for efficient design and construction—these considerations potentially benefiting unit cost. Still further, the diameter adaptive restraints may be incorporated into delivery systems offering different functional characteristics. Though the invention may have broader applicability, the exemplary variations of the invention described herein employ a stationary outer tube or sleeve and an interior wire (whether it is a corewire or another member) to actuate the restraint to draw it off of the stent to release the same upon achieving intended positioning at a target site.

Such a system includes a stent and a delivery guide for carrying the stent to a treatment site and releasing the stent at that point. In use, a physician is able to conveniently lock-down the delivery guide within the hemostatic valve of a catheter (e.g., a microcatheter or balloon catheter) if desired, and deliver a stent thus set in place.

The inner member may be a core member (i.e., filling the center of or being coaxial with the sleeve) or one of a number of inner members.

By actuating the interior member (e.g., by withdrawing the same or by a physical shortening, such as by a heat-activated shape memory plastic or alloy wire). Simple withdrawal of the inner member will deploy the stent. Yet, a more user-friendly handle could be provided. In any case, the inventive system preferably offers a simple and space efficient proximal shaft that consists of an outer tubular sleeve member and a corewire therein. Such a system is easily fit to a manipulator and/or directly manipulated by a surgeon.

Regardless of the overall delivery guide construction (an concomitant actuation), it is noted that in the inventive system the restraint only covers the implant or the implant and some distal portion of the delivery device proximal to the stent, as opposed to a system in which a simple full-length sheath is employed. The length of the restraint may be selected according to the teachings of U.S. Patent Application Attorney Docket No. CRMD-007, entitled “Sliding Restraint Stent Delivery Systems” filed on even date herewith and incorporated by reference in its entirety.

The stent or other such implant as may be employed is preferably self-expanding upon release of the restraint. Thus, full or complete placement of the stent can be achieved upon its release from the delivery device.

One embodiment of the invention operates such that a distal tip and restraint move in unison, relative to the proximal tubular member in releasing the stent from its collapsed configuration. This operation is the result of the restraint (or an intermediate connector) being attached to a core member that runs the entire length of the delivery guide, over which the stent is collapsed. Because this system is so elegant in design, it can easily be made extremely small. The device optionally includes an atraumatic tip at an end of the core member.

Another embodiment of the invention is provided in which the restraint is actuated independently of a distal tip or end of the device. In which case, the tip can be fixed relative to the sleeve and stent (axially), or it may be adjustable relative to each.

In the first instance, this result may be effected by a fixed extension section connected to the sleeve. Such devices are further detailed in U.S. Patent Application Attorney Docket No. CRMD-005, entitled “Corewire Actuated Delivery System with Distal Stent-Carrying Extension” filed on even date and incorporated by reference herein in its entirety. Alternatively, two members may be provided within the sleeve that extent proximally to the user interface. A first one of these members is in connection with the distal tip; the second one is for actuating the restraint.

Depending on the nature of the stent stop or blocker member provided to abut the stent hold it from moving axially upon withdrawal of the restraint, this two-member variation of the invention may be employed in providing the adjustable tip variation of the invention noted above. Regarding this variation of the invention, it employs a stent stop that either floats on the core wire (is slidingly received upon the wire) and interfaces with a distal end of the sleeve or that is provided by the end of the sleeve alone.

The stop may alternatively be provided by a band, shoulder or the like associated with the stent-carrying member (whether it is an extension connected to the sleeve, or a corewire extending beyond the sleeve). Still further, the stent stop or blocker may be a feature of the outer tubular member or it may be a discrete member. Alternatively, the stop member may be a multi-piece construction, include bearings, or may itself offer some form of a bearing (planar, roller, fluidic, etc.).

Generally speaking, the stent stop is adapted to allow the restraint (or a member attached thereto) to pass interior to the outer tubular member/sleeve, but not allow the stent to pass or become lodged in the moving system components.

In instances where the restraint is to be cut apart or to have portions physically separated in order facilitate drawing down from a larger outer diameter (outside the stent) to a smaller inner diameter (inside the proximal tubular member), the stop member may include separating means in the form of blades, wedges, etc. to facilitate such action Alternatively, the restraint may have an elastic or compliant quality such that it collapses to a smaller diameter when it is allowed or forced to do so. In which case, the blocker will not typically include separating means. Rather, it will simply abut the stent and provided a transition member facilitating drawing the restraint inside of the proximal tubular member.

Delivery systems and guides according to the present invention are amenable to scaling to sizes not previously achieved. Consequently, the systems may be used in lieu of a guidewire, such as in a “guidewireless” delivery approach. Still further, rather than providing an “over-the-wire” delivery system as referenced above, the present systems may be regarded as “on-the-wire” delivery systems, since—in effect—delivery is accomplished by a system in which the stent is carried by a delivery guide occupying a catheter lumen that would commonly otherwise be used to accommodate a guidewire.

Whether used in such a manner or otherwise (such as by configuring the subject systems for treating larger peripheral vessels), the present invention includes systems comprising any combination of the features described herein. Methodology described in association with the devices disclosed also forms part of the invention. Such methodology may include that associated with completing an angioplasty, bridging an aneurysm, deploying radially-expandable anchors for pacing leads or an embolic filter, or placement of a prosthesis within neurovasculature, an organ selected from the kidney and liver, within reproductive anatomy such as selected vasdeferens and fallopian tubes or other applications.

### Definitions

The term “stent” as used herein refers to any coronary artery stent, other vascular prosthesis, or other radially expanding or expandable prosthesis or scaffold-type implant suitable for the noted treatments or otherwise. Exemplary structures include wire mesh or lattice patterns and coils, though others may be employed in the present invention.

A “self expanding” stent is a scaffold-type structure (serving any of a number of purposes) that expands by its own action from a reduced-diameter configuration to an increased-diameter configuration. The “diameter” need not be circular—it may be of any open configuration. Self-expanding materials may be so by virtue of simple elastic behavior, superelastic behavior, a shape memory effect (i.e., heat-activated transformation from martinsite to austenite) or some other manner. Since the stents will remain in the subject's body, the material should be biocompatible or at least be amenable to biocompatible coating. As such, suitable self expanding stent materials for use in the subject invention include Nickel-Titanium (i.e., NiTi) alloy (e.g., NITINOL) and various other alloys or polymers.

A “wire” as used herein generally comprises a common metallic member. However, the wire may be coated or covered by a polymeric material (e.g., with a lubricious material such as TEFLON®) or otherwise. Still further, the “wire” may be a hybrid structure with metal and a polymeric material (e.g. Vectra™, Spectra™, Nylon, etc.) or composite material (e.g., carbon fiber in a polymer matrix). The wire may be a filament, bundle of filaments, cable, ribbon or in some other form. It is generally not hollow.

A “core” wire as referred to herein is a member internal to an outer member, such as a tubular member. As a core wire, the member, fills or at least substantially fills all of the interior space of the tubular member.

A “hypotube” or “hypotubing” as referred to herein means small diameter tubing in the size range discussed below, generally with a thin wall. The hypotube may specifically be hypodermic needle tubing. Alternatively, it may be wound or braided cable tubing, such as provided by Asahi Intec Co., Ltd or otherwise. As with the “wire” discussed above, the material defining the hypotube may be metallic, polymeric or a hybrid of metallic and polymeric or composite material.

A “sleeve” as referred to herein may be made of such hypotubing or otherwise. The sleeve may be a tubular member, or it may have longitudinal opening(s). It is an outer member, able to slidingly receive and hold at least a portion an inner member.

An “atraumatic tip” may comprise a plurality of spring coils attached to a tapered wire section. At a distal end the coils typically terminate with a bulb or ball that is often made of solder. In such a construction, the coils and/or solder is often platinum alloy or another radiopaque material. The coils may also be platinum, or be of another material. In the present invention, the wire section to which the coils are attached may be tapered, but need not be tapered. In addition, alternate structures are possible. For instance, molding or dip-coating with a polymer may be employed. In one example, the atraumatic tip may comprise a molded tantalum-loaded 35 durometer Pebax™ tip. However constructed, the atraumatic tip may be straight or curved, the latter configuration possibly assisting in directing or steering the delivery guide to a desired intravascular location.

To “connect” or to have or make a “connection” between parts refers to fusing, bonding, welding (by resistance, laser, chemically, ultrasonically, etc), gluing, pinning, crimping, clamping or otherwise mechanically or physically joining, attaching or holding components together (permanently or temporarily).

### BRIEF DESCRIPTION OF THE DRAWINGS

Each of the figures diagrammatically illustrates aspects of the invention. Of these:

FIG. 1 shows a heart in which its vessels may be the subject of one or more angioplasty and stenting procedures;

FIG. 2 shows an expanded stent cut pattern as may be used in producing a stent for use in the present invention;

FIGS. 3A-3L illustrate stent deployment methodology to be carried out with the subject delivery guide member;

FIG. 4 provides an overview of the inventive system;

FIGS. 5 show general aspects of a diameter adaptive restraint according to the present invention;

FIGS. 6A-6D include partial side-sectional views showing the overall operation of each of five variations of the invention; FIG. 6E includes cross-sectional views illustrating various pass-through connections between the device sleeve and a member extending therefrom.

FIGS. 7A and 7B provide flattened-out views of restraints as may be used in the invention;

FIGS. 8A and 8B show views as in FIGS. 7A and 7B of another restraint in its larger diameter, and its reduced diameter configurations;

FIG. 9 is a partial side view of planar-bearing type stent stop or blocker member;

FIG. 10 is a perspective view of a ball-bearing type stent stop;

FIG. 11 is a partial side-sectional view of a diameter adaptive restraint system employing a fluidic bearing;

FIG. 12A is a side-sectional view of a stent stop in the form of a roller bearing; FIG. 1 2B is a partial side sectional view of a delivery device employing the stop member shown in FIG. 12A; and

FIG. 13A is an end view of a delivery device proximal to a stent including restraint cutter and/or stent stop features; FIG. 13B is a side view of the structure FIG. 13A, with an optional floating stent stop member.

Variation of the invention from the embodiments pictured is, of course, contemplated.

### DETAILED DESCRIPTION OF THE INVENTION

Before the present invention is described in detail, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “and,” “said” and “the” include plural referents unless the context clearly dictates otherwise. It is further noted that the claims may be drafted to exclude any optional element. As such, this statement is intended to serve as antecedent basis for use, of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs.

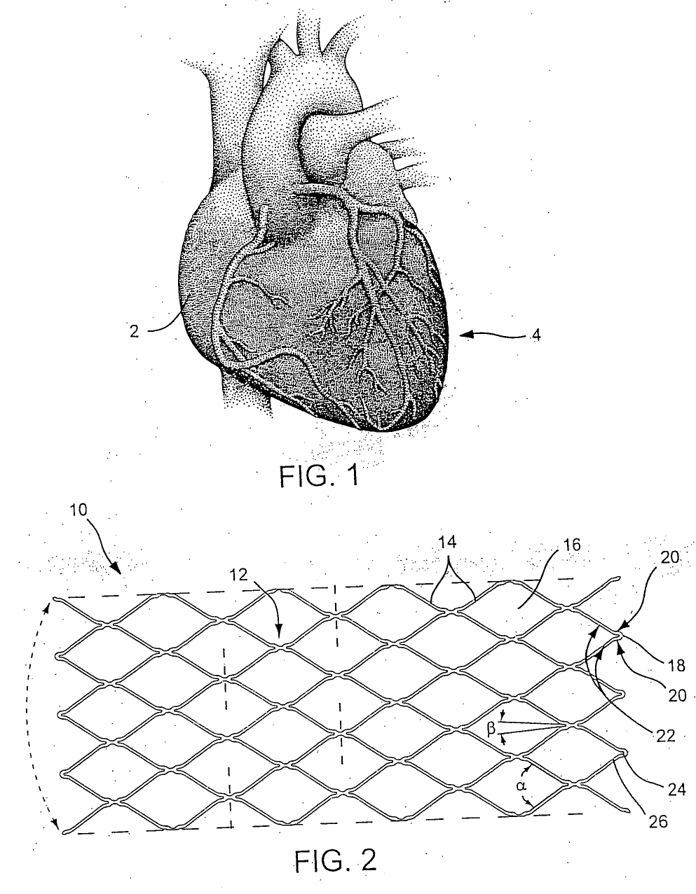

Turning now to FIG. 1, it shows a heart 2 in which its vessels may be the subject of one or more angioplasty and/or stenting procedures. To date, however, significant difficulty or impossibility is confronted in reaching smaller coronary arteries 4. If a stent and a delivery system could be provided for accessing such small vessels and other difficult anatomy, an additional 20 to 25% coronary percutaneous procedures could be performed with such a system. Such a potential offers opportunity for huge gains in human healthcare and a concomitant market opportunity in the realm of roughly $1 billion U.S. dollars—with the further benefit of avoiding loss of income and productivity of those treated.

Features of the present invention are uniquely suited for a system able to reach small vessels (though use of the subject systems s not limited to such a setting.) By “small” coronary vessels, it is meant vessels having a inside diameter between about 1.5 or 2 and about 3 mm in diameter. These vessels include, but are not limited to, the Posterior Descending Artery (PDA), Obtuse Marginal (OM) and small diagonals. Conditions such as diffuse stenosis and diabetes produce conditions that represent other access and delivery challenges which can be addressed with a delivery system according to the present invention. Other extended treatment areas addressable with the subject systems include vessel bifurcations, chronic total occlusions (CTOs), and prevention procedures (such as in stenting vulnerable plaque).

Assuming a means of delivering one or more appropriately-sized stents, it may be preferred to use a drug eluting stent in such an application to aid in preventing restenosis. However, bare-metal stents may be employed in the present invention. The present invention is advantageously employed with self-expanding stents. However, the teachings herein may be adapted for application in the context of balloon-expandable stents.

In any case, features of the present invention are provided in order to hold an implant (e.g., a stent) to be delivered in an access or deployment configuration, after which, the implant assumes its deployed or expanded configuration. Hold-down features may restrain a stent under compressive forces, whereupon release, the stent “springs” open. Alternatively, the stent (or other implant) may simply be secured to the delivery member, where some other mechanism is used to open the stent (e.g., ceasing a flow of chilled saline, thereby allowing a shape memory devices (e.g., NiTi) to warm in order that a material phase change from martinsite to austenite will cause the stent to open).

While some might argue that the particular role and optimal usage of self expanding stents has yet to be defined, they offer an inherent advantage over balloon expandable stents. The latter type of devices produce “skid mark” trauma (at least when delivered uncovered upon a balloon) and are associated with a higher risk of end dissection or barotraumas caused at least in part by high balloon pressures and related forces when deforming a balloon-expandable stent for deployment.

Yet, with an appropriate deployment system, self-expanding stents may offer one or more of the following advantages over balloon-expandable models: 1) greater accessibility to distal, tortuous and small vessel anatomy—by virtue of decreasing crossing diameter and increasing compliance relative to a system requiring a deployment balloon, 2) sequentially controlled or “gentle” device deployment, 3) use with low balloon pre-dilatation (if desirable) to reduce barotraumas, 4) strut thickness reduction in some cases reducing the amount of “foreign body” material in a vessel or other body conduit, 5) opportunity to treat neurovasculature—due to smaller crossing diameters and/or gentle delivery options, 6) the ability to easily scale-up a successful treatment system to treat larger vessels or vice versa, 7) a decrease in system complexity, offering potential advantages both in terms of reliability and system cost, 8) reducing intimal hyperplasia, and 9) conforming to tapering anatomy—without imparting complimentary geometry to the stent (though this option exists as well).

At least some of these noted advantages may be realized using a stent 10 as shown in FIG. 2 in connection with the subject deployment system described in further detail below. Naturally, other stent configurations might be used instead. However, the one pictured is well suited for use in small vessels. It may be collapsed to an outer diameter of about 0.018 inch (0.46 mm), or even smaller to about 0.014 inch (0.36 mm)—including the restraint/joint used—and expand to a size (fully unrestrained) between about 1.5 mm (0.059 inch) or 2 mm (0.079 inch) or 3 mm (0.12 inch) and about 3.5 mm (0.14 inch).

In use, the stent will be sized so that it is not fully expanded when fully deployed against the wall of a vessel in order that it will provide a measure of radial force thereto. The force will secure the stent and offer potential benefits in reducing intimal hyperplasia and vessel collapse or even pinning dissected tissue in apposition.

The stent employed in connection with the subject delivery system preferably comprises NiTi that is superelastic at room temperature and above. Also, it is preferably electropolished. The stent may be a drug eluting stent (DES). Such drug can be directly applied to the stent surface(s), or introduced into an appropriate matrix.

In a 0.014 inch delivery system (one in which the maximum nominal outer diameter of the stent/coating and guide member/restraint have a diameter that does not exceed 0.014 inch), the thickness of the NiTi is about 0.0025 inch (0.64 mm) for a stent adapted to expand to 3.5 mm. Such a stent is designed for use in a 3 mm vessel or other body conduit, thereby providing the desired radial force in the manner noted above. Further information regarding radial force parameters in coronary stents may be noted in the article, “Radial Force of Coronary Stents: A Comparative Analysis,” Catheterization and Cardiovascular Interventions 46: 380-391 (1999), incorporated by reference herein in its entirety.

As for the stent that may be employed, an optional expanded stent cut pattern 10 is shown in FIG. 2. In one manner of production, the stent is laser (or Electrical Discharge Machining, i.e., EDM) cut from round NiTi tubing, with the flattened-out pattern shown wrapping around the tube as indicated by dashed lines. In such a procedure, the stent is preferably cut in its fully-expanded shape. By initially producing the stent to full size, the approach allows cutting finer details in comparison to simply cutting a smaller tube with slits and then heat-expanding/annealing it into its final (working) diameter. Avoiding post-cutting heat forming also reduces production cost.

Regarding the finer details of the subject stent, necked down bridge or junction sections 12 are provided between adjacent struts 14, wherein the struts define a lattice of closed cells 16. The ends 18 of the cells are preferably rounded-off so as to be atraumatic. To increase stent conformability to tortuous anatomy, the bridge sections can be strategically separated or opened as indicated by broken line. To facilitate such tuning of the stent, the bridge sections are sufficiently long so that fully rounded ends 18 may be formed internally to the lattice just as shown on the outside of the stent if the connection(s) is/are severed to separate adjacent cells 16.

The advantage of the double-concave profile of each strut bridge or junction section 12 is that it reduces material width (relative to what would otherwise be presented by a parallel side profile) to improve trackability and conformability of the stent within the subject anatomy while still maintaining the option for separating/breaking the cells apart.

Further optional features of stent 10 are employed in the cell end regions 18 of the design. Specifically, strut ends 20 increase in width relative to medial strut portions 22. Such a configuration results in a majority of bending (during collapse of the stent) occurring along the length of the struts rather than at the corners of the cells. Longer struts to allow for lower stresses within the stent (and, hence, possibility for higher compression ratios). Shorter struts allow for greater radial force (and concomitant resistance to a radially applied load) upon deployment.

In order to provide a stent that collapses as much as possible (to solid or near-solid structure, such as shown in the fully-loaded systems of the figures) accommodation is made for the stiffer strut ends 20 provided in the design shown in FIG. 2. Namely, the gap 24 between the strut ends 22 is set at a smaller angle as if the stent were already partially collapsed in that area. Thus, the smaller amount of angular deflection that occurs at ends 20 will bring the sections parallel (or nearly so) when the strut medial portions 22 are so-arranged. Radiused sections 26 provide a transition from a medial strut angle a (ranging from about 85 degrees to about 60 degrees) to an end strut angle β (ranging from about 30 to about 0 degrees). In addition, it is noted that gap 24 and angle β may actually be configured to completely close prior to fully collapsing angle α. The value of doing so would be to limit the strains (and hence, stresses) at the strut ends 22 and cell end regions 18 by providing a physical stop to prevent further strain.

By utilizing a design that minimizes strain, very high compression ratios of the stent may be achieved. Compression ratios (from a fully expanded outside diameter to compressed outside diameter—expressed in those terms used by physicians) of as much as 3.5 mm: 0.014 inch (about 10×) are possible—with or without a drug coating and/or restraint used. Compression ratios of 3.0 mm: 0.014 inch (about 8.5×), 3.5 mm: 0.018 inch (about 7.5×), 3.0 mm: 0.018 inch (about 6.5×), 2.5 mm: 0.014 inch (about 7×), 2.5 mm: 0.018 inch (about 5.5×), 2.0 mm: 0.014 inch (about 5.5×), 2.0 mm: 0.018 inch (about 4.5×) offer utility not heretofore possible with existing systems as well.

These selected sizings (and expansion ratios) correspond to treating 1.5 to 3.0 mm vessels by way of delivery systems adapted to pass through existing balloon catheter and microcatheter guidewire lumen. In other words, the 0.014 inch and 0.018 inch systems are designed to corresponding common guidewire sizes. The system may also be scaled to other common guidewire sizes (e.g., 0.22 inch/0.56 mm or 0.025 inch/0.64 mm) while offering advantages over known systems.

While designing the delivery systems to have a crossing profile corresponding to common guidewire sizes, especially for full-custom systems, intermediate sizes may be employed. Still further, it is contemplated that the system sizing may be set to correspond to French (FR) sizing. In that case, system sizes contemplated range at least from 1 to 1.5 FR, whereas the smallest know balloon-expandable stent delivery systems are in the size range of about 3 to about 4 FR.

At least when produced at the smallest sizes (whether in a even/standard guidewire or FR size, or otherwise), the system enables a substantially new mode of stent deployment in which delivery is achieved through an angioplasty balloon catheter or small microcatheter lumen. Further discussion and details of “through the lumen” delivery is presented in the above-referenced “Balloon Catheter Lumen Based Stent Delivery Systems” patent application.

In “small vessel” cases or applications (where the vessel to be treated has a diameter up to about 3.0 mm), it may also be advantageous to employ a stent delivery system sized at between about 0.022 to about 0.025 inch in diameter. Such a system can be used with catheters compatible with 0.022 inch diameter guidewires.

While such a system may not be suitable for reaching the very smallest vessels, in reaching the larger of the small vessels (i.e., those having a diameter of about 2.5 mm or larger), even this variation of the invention is quite advantageous in comparison to known systems. By way of comparison, the smallest known over-the-guidewire delivery system (the “Pixel” system—produced by Guidant) that is adapted to treat vessels between 2 and 2.5 mm has a crossing profile of 0.036 inch (0.91 mm). A system described in U.S. Patent Publication No. 2002/0147491 for treating small vessels is purported to be capable of being made as small as 0.026 inch (0.66 mm) in diameter.

With respect to the Pixel and '491 systems, however, it must be appreciated that a further decrease in stent size may be practically impossible in view of materials limitations and functional parameters of the stent. Instead, the present invention offers a different paradigm for delivery devices and stents that are scalable to the sizes noted herein.

By virtue of the approaches taught herein, it is feasible to design system diameters to match (or at least nearly match) common guidewire size diameters (i.e., 0.014, 0.018 and 0.022 inch) for small vessel delivery applications. As noted above, doing so facilitates use with compatible catheters and opens the possibility for methodology employing the same as elaborated upon below and in the above-referenced “Balloon Catheter Lumen Based Stent Delivery Systems” patent application.

Of further note, it may be desired to design a variation of the subject system for use in deploying stents in larger, peripheral vessels, bilary ducts or other hollow body organs. Such applications involve a stent being emplaced in a region having a diameter from about 3.5 to about 13 mm (0.5 inch). In this regard, the scalability of the present system, again, allows for creating a system adapted for such use that is designed around a common wire size. Namely, a 0.035 to 0.039 inch (3 FR) diameter crossing profile system is advantageously provided in which the stent expands (unconstrained) to a size between about roughly 0.5 mm and about 1.0 mm greater than the vessel or hollow body organ to be treated. Sufficient stent expansion is easily achieved with the exemplary stent pattern shown in FIG. 2.

Again, as a matter of comparison, the smallest delivery systems known to applicants for stent delivery in treating such larger-diameter vessels or biliary ducts is a 6 FR system (nominal 0.084 inch outer diameter), which is suited for use in an 8 FR guiding catheter. Thus, even in the larger sizes, the present invention affords opportunities not heretofore possible in achieving delivery systems in the size range of a commonly used guidewire, with the concomitant advantages discussed herein.

Several known stent delivery systems are compatible with (i.e., may be delivered over) common-sized guides wires ranging from 0.014 inch to 0.035 inch (0.89 mm). Yet, none of the delivery systems are themselves known to be so-sized.

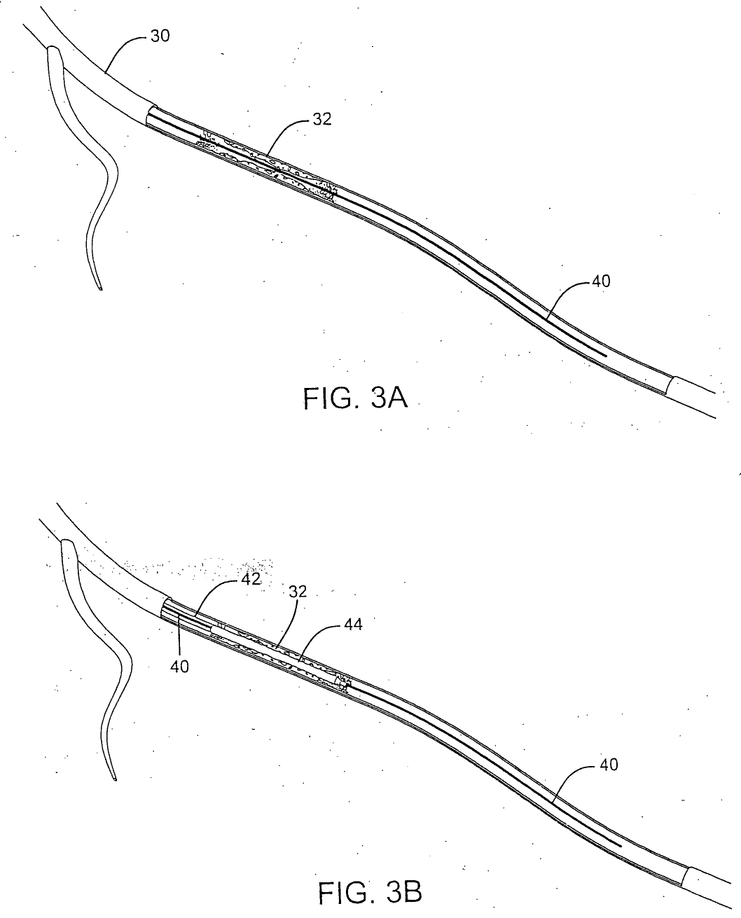

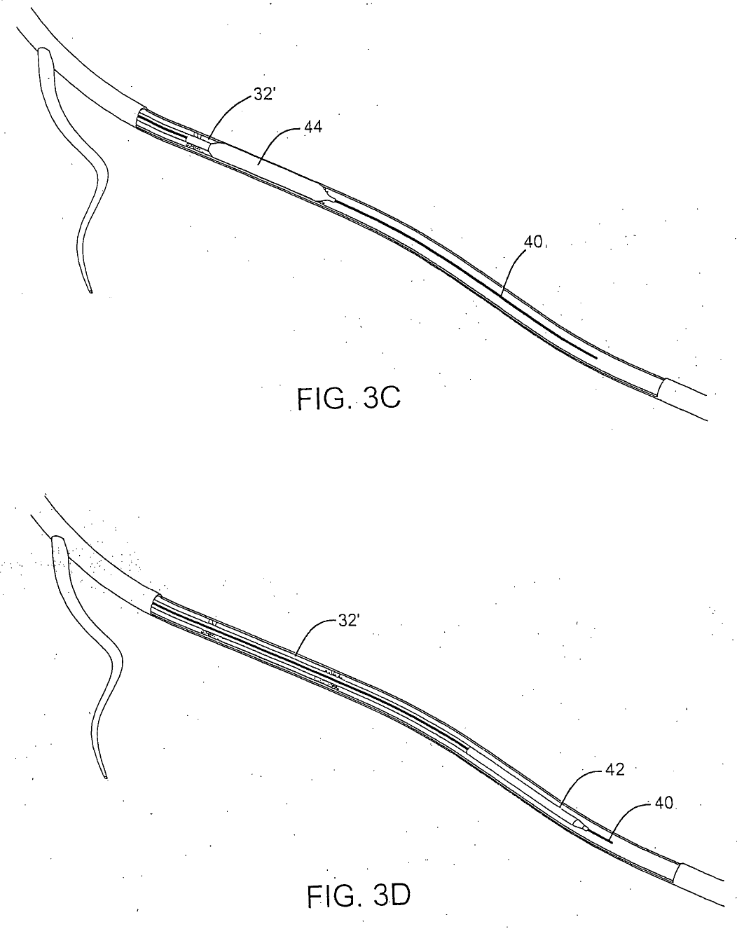

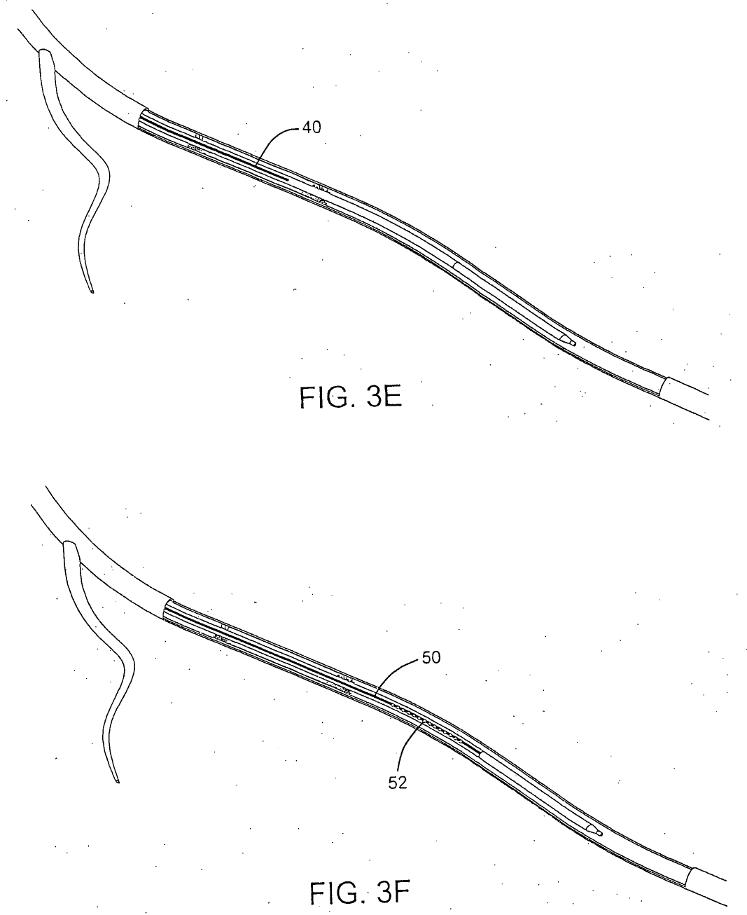

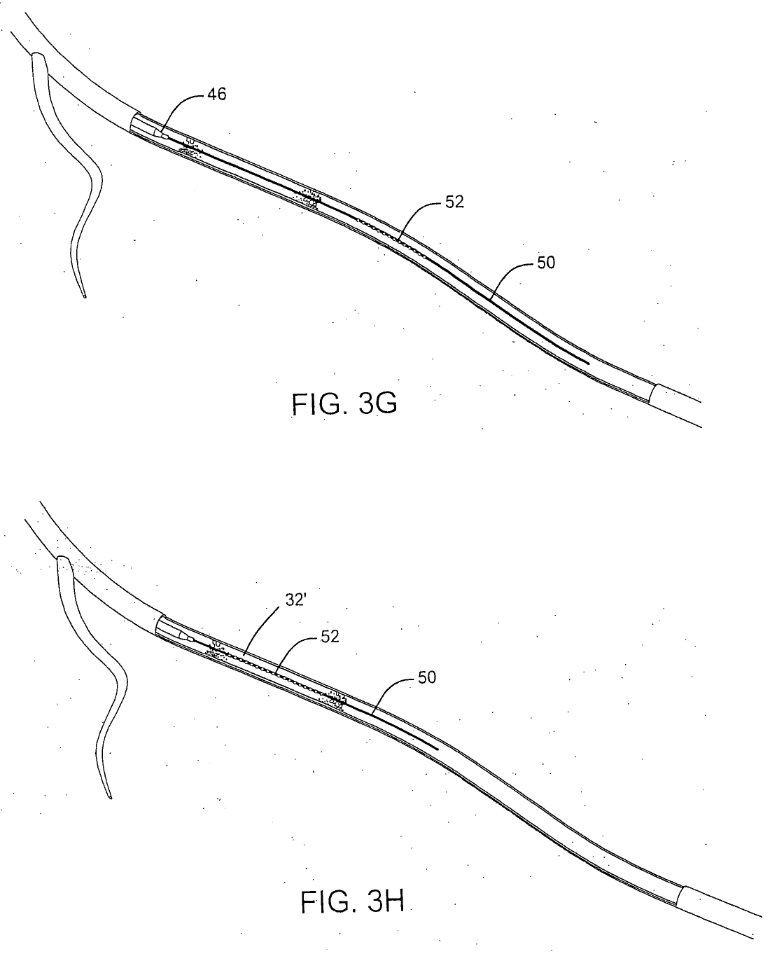

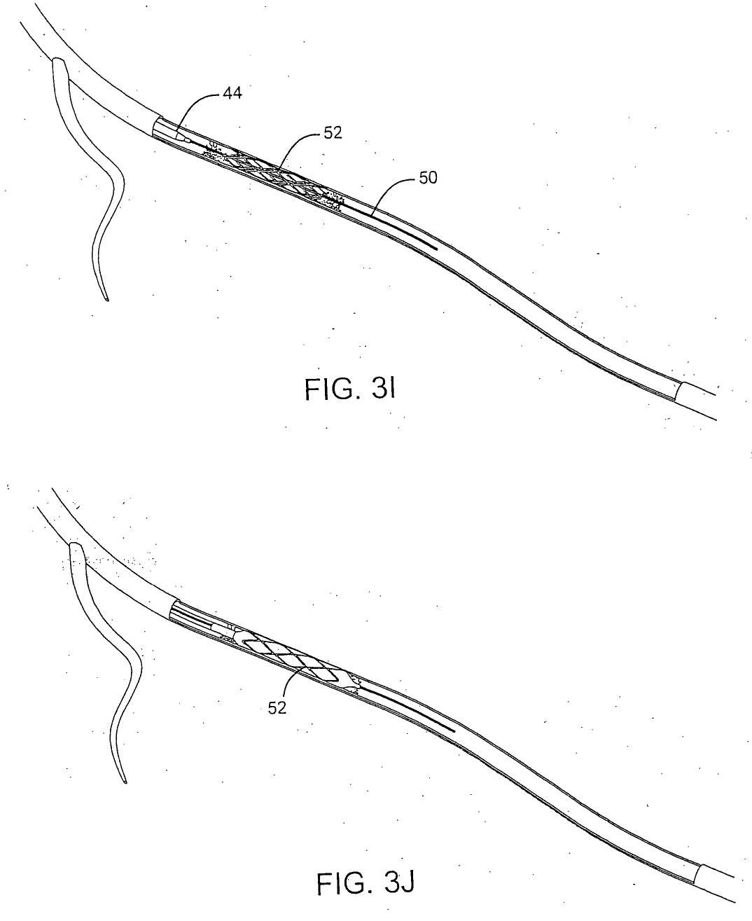

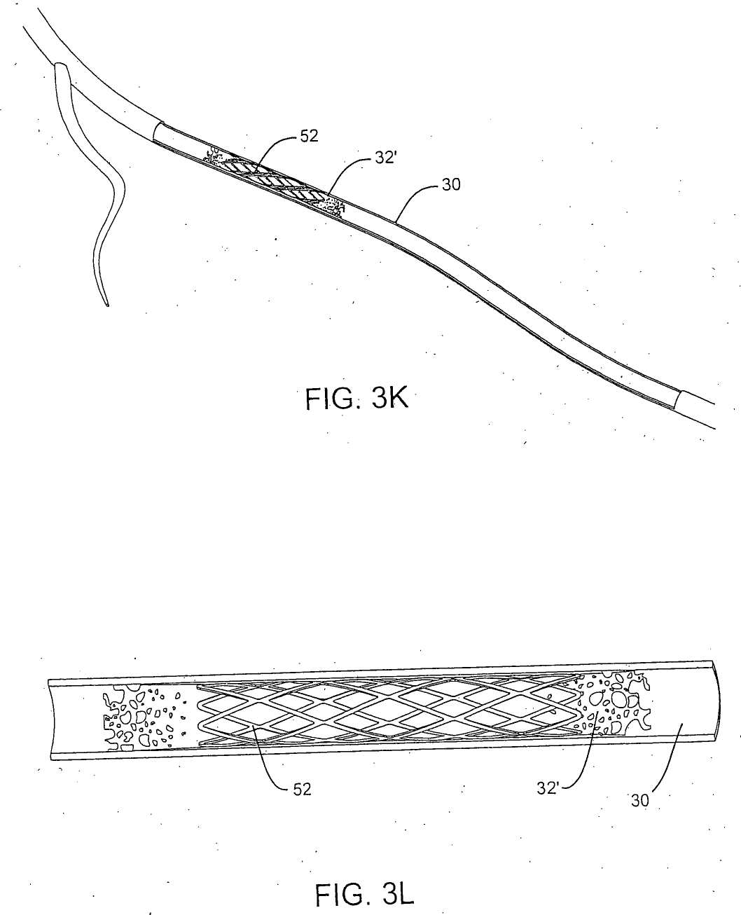

As for the manner of using the inventive system as optionally configured, FIGS. 3A-3L illustrate an exemplary angioplasty procedure. Still, the delivery systems and stents or implants described herein may be used otherwise—especially as specifically referenced herein.

Turning to FIG. 3A, it shows a coronary artery 30 that is partially or totally occluded by plaque at a treatment site/lesion 32. Into this vessel, a guidewire 40 is passed distal to the treatment site. In FIG. 3B, a balloon catheter 42 with a balloon tip 44 is passed over the guidewire, aligning the balloon portion with the lesion (the balloon catheter shaft proximal to the balloon is shown in cross section with guidewire 40 therein).

As illustrated in FIG. 3C, balloon 44 is expanded (dilatated or dialated) in performing an angioplasty procedure, opening the vessel in the region of lesion 32. The balloon expansion may be regarded as “predilatation” in the sense that it will be followed by stent placement (and optionally) a “postdilataton” balloon expansion procedure.

Next, the balloon is at least partially deflated and passed forward, beyond the dilate segment 32′ as shown in FIG. 3D. At this point, guidewire 40 is removed as illustrated in FIG. 3E. It is exchanged for a delivery guide member 50 carrying stent 52 as further described below. This exchange is illustrated in FIGS. 3E and 3F.

However, it should be appreciated that such an exchange need not occur. Rather, the original guidewire device inside the balloon catheter (or any other catheter used) may be that of item 50, instead of the standard guidewire 40 shown in FIG. 3A. Thus, the steps depicted in FIGS. 3E and 3F (hence, the figures also) may be omitted. In addition, there maybe no use in performing the step in FIG. 3D of advancing the balloon catheter past the lesion, since such placement is merely for the purpose of avoiding disturbing the site of the lesion by moving a guidewire past the same.

FIG. 3G illustrates the next act in either case. Particularly, the balloon catheter is withdrawn so that its distal end 46 clears the lesion. Preferably, delivery guide 50 is held stationary, in a stable position. After the balloon is pulled back, so is delivery device 50, positioning stent 52 where desired. Note, however, that simultaneous retraction may be undertaken, combining the acts depicted in FIGS. 3G and 3H. Whatever the case, it should also be appreciated that the coordinated movement will typically be achieved by virtue of skilled manipulation by a doctor viewing one or more radiopaque features associated with the stent or delivery system under medical imaging.

Once placement of the stent across from dilated segment 32′ is accomplished, stent deployment commences. The manner of deployment is elaborated upon below. Upon deployment, stent 52 assumes an at least partially expanded shape in apposition to the compressed plaque as shown in FIG. 31. Next, the aforementioned postdilatation may be effected as shown in FIG. 3J by positioning balloon 44 within stent 52 and expanding both. This procedure may further expand the stent, pushing it into adjacent plaque—helping to secure each.

Naturally, the balloon need not be reintroduced for postdilatation, but it may be preferred. Regardless, once the delivery device 50 and balloon catheter 42 are withdrawn as in FIG. 3K, the angioplasty and stenting procedure at the lesion in vessel 30 is complete. FIG. 3L shows a detailed view of the emplaced stent and the desired resultant product in the form of a supported, open vessel.

In the above description, a 300 cm extendable delivery system is envisioned. Alternatively, the system can be 190 cm to accommodate a rabid exchange of monorail type of balloon catheter as is commonly known in the art. Of course, other approaches may be employed as well.

Furthermore, other endpoints may be desired such as implanting an anchoring stent in a hollow tubular body organ, closing off an aneurysm, delivering a plurality of stents, etc. In performing any of a variety of these or other procedures, suitable modification will be made in the subject methodology. The procedure shown is depicted merely because it illustrates a preferred mode of practicing the subject invention, despite its potential for broader applicability.

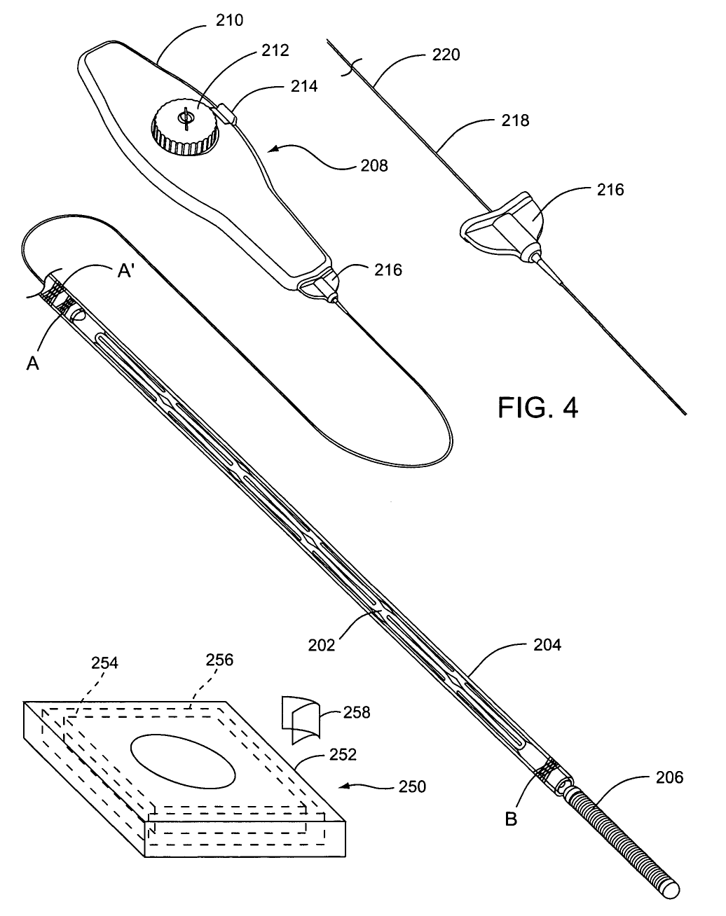

A more detailed overview of the subject delivery systems is provided in FIG. 4. Here, a delivery system 200 is shown along with a stent 202 held onto a delivery guide in a collapsed configuration. A restraint 204 is provided over and around the stent. The restraint may fully surround the stent or only subtend a partial circumference, it may be split, splittable, comprise a plurality of member or be otherwise provided around the stent to hold or restrain it in a collapsed profile.

Regarding the overall delivery guide, however, it preferably comprises an atraumatic distal tip 206 of one variety or another. On the other end of the delivery device, a custom handle 208 is preferably provided.

The handle shown is adapted for rotable actuation by holding body 210, and turning wheel 212. It may include a lock 214. Furthermore, a removable interface member 216 facilitates taking the handle off of the delivery system proximal end 218. The interface will be lockable with respect to the body and preferably includes internal features for disengaging the handle from the delivery guide. Once accomplished, it will be possible to attach or “doc” a secondary length of wire 220 on the delivery system proximal end, allowing the combination to serve as an “exchange length” guidewire, thereby facilitating changing-out the balloon catheter or performing another procedure. Alternatively, the wire may be an exchange-length wire.

FIG. 4 also shows packaging 250 containing at least one coiled-up delivery systems 200. When a plurality of such systems are provided (in one package or by way of a number of packages held in stock), they are typically configured in support of a methodology where an appropriate one is picked to reach a target site and deploy a stent without unintended axial movement of the same as per the methodology of U.S. Patent Application Attorney Docket No. CRMD-007 entitled, “Sliding Restraint Stent Delivery Systems” filed on even date herewith and incorporated by reference in its entirety.

Thus, the packaging may serve the purpose of providing a kit or panel of differently configured delivery devices. In the alternative, the packaging may be configured as a tray kit for a single one of the delivery systems. Either way, packaging may include one or more of an outer box 252 and one or more inner trays 254, 256 with peel-away coverings as is customary in packaging of disposable products provided for operating room use. Naturally, instructions for use can be provided therein. Such instructions may be printed product or be provided in connection with another readable (including computer-readable) medium. The instructions may include provision for basic operation of the subject devices and/or the selection methodology.

Regarding the specifics of the restraint employed in the delivery device, it is one in which at least a length of it is received within the body of the device after beginning at a larger diameter (possibly over the stent, or proximal thereto) and collapsing (by folding, overlapping, compressing or other means) to a smaller diameter to fit, for example, within the body of the delivery device.

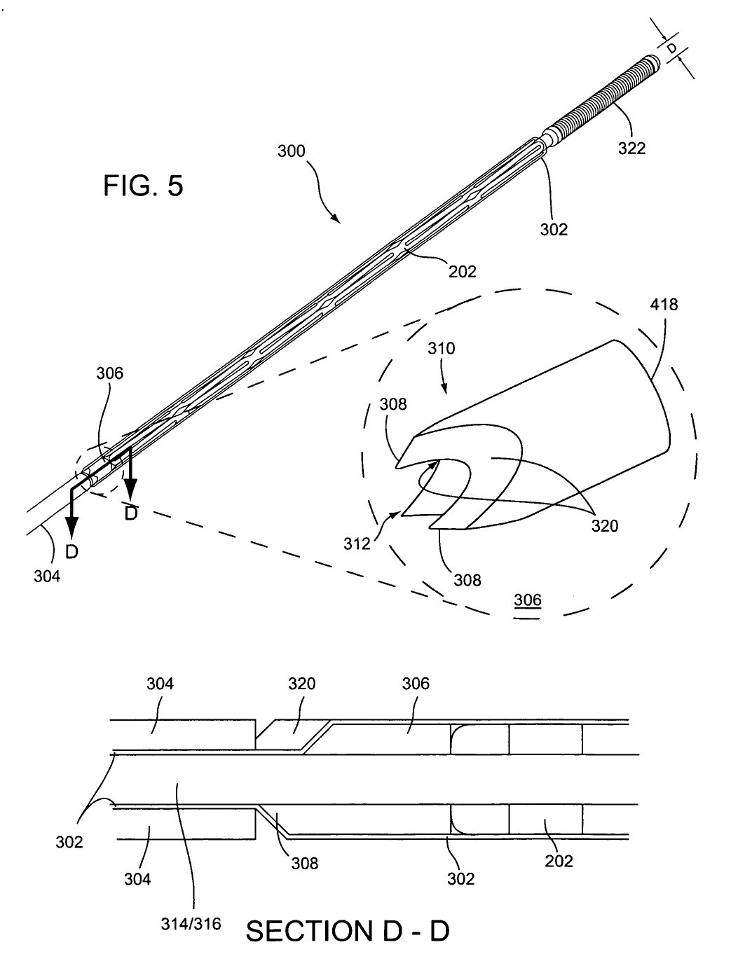

One variation of the restraint is shown in FIG. 5. The delivery guide, of which a distal portion 300 is pictured, operates in a manner such that its crossing profile or delivery diameter D does not increase during, or after, stent 202 release.

In short, the device releases the stent when restraint 302 is pulled back into sleeve 304 sufficiently to move off of the stent. Such action may be facilitated by using a pre-split restraint or restraint sections. Alternatively, the restraint may be separated (e.g. along a perforation line or lines). This may be facilitated by a wedge type member. Still further, the restraint may be cut into sections. Moreover, the restraint may itself be collapsible in nature.

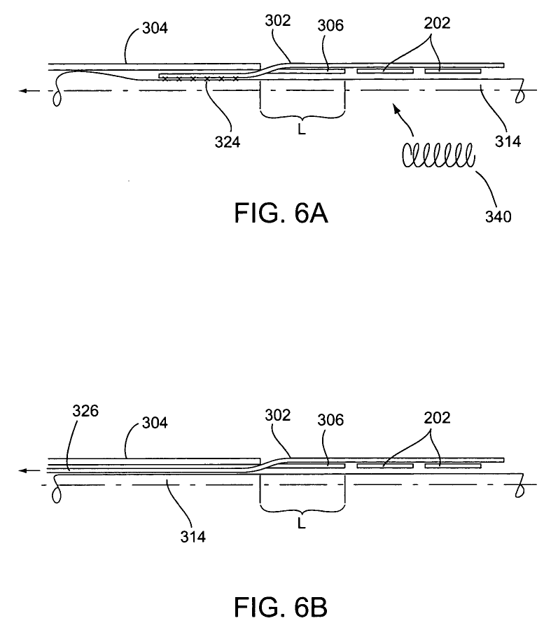

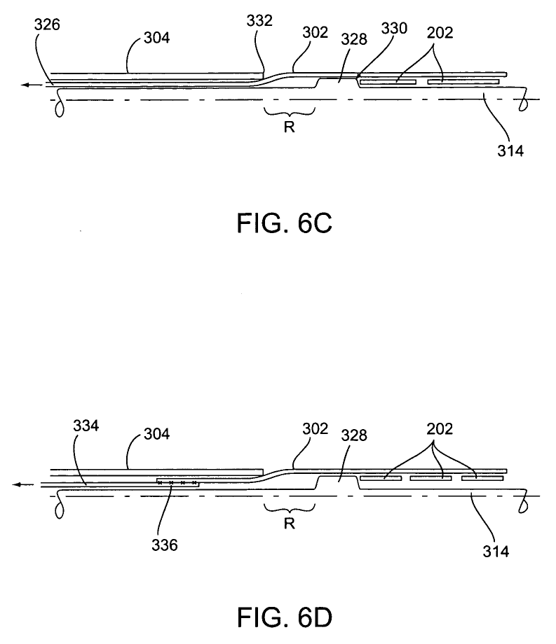

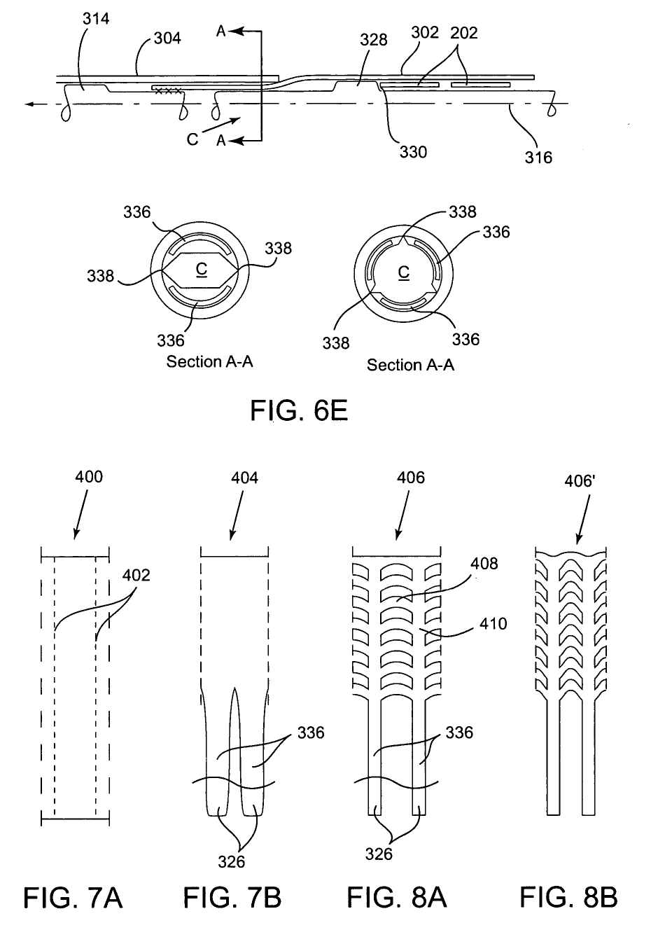

The device in FIG. 5 is configured for such operation in which the restraint is cut or split apart. To facilitate this action, a stent stop or interface member 306 is provided. In the enlarged detail of the same, one can clearly see blade portions 308. In this example, the blades are formed by cutting tubing on a bias at a proximal end 310. The lumen 312 defined by the tubing accepts either the inner/core member running the full length of the device 314 (as in the embodiments in FIGS. 6A and 6B) or an extension wire 316 (as in the embodiment shown in FIG. 6E). A distal end 318 of the interface member provides a stop section for the stent.

As to the specific manner of operation, Section D-D is provided to help explain such operation. In this sectional view, restraint 302 is shown diving down from outside of the stent 202 to within the sleeve 304. The sections of the restraint are cut or separated into pass-through recesses 320.

In some variations of the invention (as detailed further below), the inner member that is actuated to withdraw the restraint may be an extension of the restraint itself, a tubular member connected thereto that runs the length of the system or it may be a core member 314. If it is a core member then (as stated above) it may be desirable to include an extension wire distal thereto.

In any case, various restraint actuation options are specifically illustrated in FIGS. 6A-6E. In FIG. 6A, the device is corewire actuated. Restraint 302 is show directly attached, for example, by adhesive 324 an inner member 314 (pictured in this case as a corewire providing column strength the system and aiding in its noted desired functional characteristics in terms—such as in terms of torqueability and directability to a target site). Note, however, that an intermediate connector or bridge section can be employed. The same is true of the other variations discussed below as well. Another option is to include a spring element 340 or other elastically or plastically extensible (possibly recoverable) element within the core member 314. That is to say, the core member may be broken into proximal and distal pieces, with a spring connected between each. Providing such a system would allow withdrawal of the restraint, while leaving the distal tip of the device secured (if it happens to be lodged) with anatomy distal of the delivery device. Such a system offers a “semi-fixed” tip device. If the tip does indeed intended to remain stationary upon withdrawal of the inner member proximal end, then it may be desired to include locking features between the spring and the inner member portions to prevent inadvertent separation, or additionally allow the pieces to be put back together for ultimate withdrawal of the delivery guide for the subject anatomy.

Regardless, stent stop 306 is provided between a distal end of sleeve 304 and the stent 202. Various options for the stop are discussed further below.

In FIG. 6B, restraint 302 is actuated by an extension section 326. The extension, which is, itself an inner member to sleeve, may be formed from the same piece of material as the restraint, by drawing down tubing (polymer or metal hypotube), or be provided, again, by a member connected thereto.

Regarding the variations of the invention in FIGS. 6A and 6B it is worth noting that stop member 306 slides freely over core wire 314 (or vice versa). As such, after release of the stent, it is important to ensure that the stop is not inadvertently released from the delivery device as well. Accordingly, various safety precautions may be taken, including providing a limit in the handle to prevent the restraint from being withdrawn from over the stop, a long length stop to increase its frictional engagement with the restraint, etc. In any case, a longer stop body length (“L”) will tend to avoid off-center loads preventing free sliding of the core member within the stop member. Practical engineering guidance suggest a length three times that of the sliding engagement diameter, though other lengths (lesser or greater) may be employed.

While both of the system actuation approaches in FIGS. 6A and 6B employ “floating” stop members, it should further be noted that the variation in FIG. 6A (when a tip is provided) is a moving tip type device, while that of FIG. 6B is not necessarily so. The variation of the invention in FIG. 6B can be configured for fixed-tip use or as an adjustable position tip (relative to the sleeve) depending on whether a proximal end of core wire 314 is fixed stationary relative to the sleeve, or is allowed axial freedom (by a handle or otherwise).

While sharing the same mode of restraint actuation (namely via an extension section 326 from the restraint), the variation of the invention in FIG. 6C will often be configured as a fixed position tip type device. In which case, core member 314 cannot be adjusted axially. Note, however, that while the device cannot be configured so that the core member is proximally adjustable relative to the restraint in the position shown (since ring or shoulder 328, with its stop surface 330 for the stent would push the stent forward out of the restraint if moved in that direction), corewire 314 may be configured to have a range of adjustability (“R”) between the stent 202 and distal end 320 of sleeve 304. The same holds true for the variation of the invention in FIG. 6D—which is actuated like the embodiments of FIGS. 6B and 6C, with the exception that a separate elongate member 334 is attached to the restraint to provide for actuation at a proximal end instead of an extension of the restraint.

This elongate member may take the same form as the extension section 326 (i.e., it may be tubular, flat ribbon, a wire or wire-like). However, it offers additional material and constructional possibilities for the system, though the overlapped bonding section 336 may utilize valuable space. Yet, accommodation may be made for the same by way of variously undercutting and/or tapering core member 314.

The variation of the invention shown in FIG. 6E is also presented in U.S. Patent Application Attorney Docket No. CRMD-005, referenced above. In this variation, corewire 314 is slidingly received by a sleeve 304. Stent 202 is held in a collapsed configuration by a restraint 302 over an extension wire/member 316 connected to sleeve 304. A shoulder section 328 having a distal stent stop surface 330 is provided on the extension.

Connection options between the extension member and the sleeve are shown in Sections A-A. The sections show the connection sections (“C”), as well as the manner in which bridge sections 336 (in connection with the corewire 314 and restraint 302) pass by the same. Furthermore, blade or wedge sections 338 are provided for separating the restraint progressively (in a preferred embodiment) into bridge the bridge sections 336 upon withdrawal of the restraint to release the stent.

In addition, as noted above, other embodiments of the invention that advantageously employ the diameter adaptive restraint of the present invention are possible. The ability of the restraint to collapse in size and be hidden-away within the body of the delivery device (sometimes merely replacing the space where the stent was carried distally on the delivery device anyway) can be highly advantageous. The systems can be simple to construct and very space efficient and cost effective.

As for various options for the restraint, some are illustrated in FIGS. 7A-7C. FIG. 7A shows the simplest form of restraint. The figure portrays an unrolled tube 400. It may be an elastomeric material in order that is conforms to a reduced diameter within the delivery device sleeve. Alternatively, it is advantageously a plastic such as Polyimide or PET. When made of a material not collapsible, it may be of such a sort that axial wrinkles formed will adequately reduce its diameter to fit within the sleeve receiving the same.

When in reducing the diameter of a section of the restraint from one diameter to a another diameter such that it is “substantially reduced”, the reduction in diameters is greater than that experienced in withdrawing a know sheath off of a stent (the amount is of reduction is greater than that due to the elasticity inherent to known systems). In this respect, the diameter reduction may be at least about 2% or 5%, between about 5% and about 10%, about 10% and a about 20%, about 20% and 30%, even up to about 50%. Such diameter reduction may occur by elastic recovery or deformation by an outer member, or even plastic deformation.

In one mode of the invention, the diameter change may be less than noted. However, the change will be effected by urging the restraint portion to a reduced diameter by a member external thereto.. The motivating member may take the form of the sleeve or the interface or stop member. Even a small reduction in diameter under such circumstances, can offer significant space-savings advantages, for example, as the restraint fills space left vacant by stent progressively as it is released.

In any case, where the material is not able to assume a reduced diameter by uniform elastic recovery or some form of deformation, the material may be cut apart so that portions will fold or overlap over each other (optionally in a concentric fashion). To facilitate such action, scoring or perforations 402 may be provided—such as by laser machining or another procedure. These sections will correspond to where the restraint is broken apart to provide ridge sections to pass by a connection C, such as in the variation of the device shown in FIG. 5, and 6E—and, optionally, those of FIGS. 6A and 6B where stop 306 includes restraint separating features.

In order to facilitate such separation of members, as well as proximal connection, restraint 404 includes preformed bridge sections 336. In fact, the “legs” may extend so far back as to the proximal end of the device, thereby providing extensions 326 as discussed above. Restraint 404 may be of a similar construction to restraint 400.

However, restraint 406 in FIGS. 8A and 8B is quite different. While optionally presenting the bridge/restraint feature options as restraint 404, restraint 406 is made to collapse by virtue of its geometry. Struts (as in a stent) 408 are provided that pack-down to support a reduced diameter configuration 406′ as shown in FIG. 8B. Runner or rib sections 410 may be provided to ensure that the overall length of the restraint does not increase upon collapse and also to aid in transmitting axial force. Struts 408 are configured so as not to catch on the stent upon withdrawal. Thus, a sort of scale-like configuration as shown may be desirable where any points or curvature of the members face in one direction. Restraint 406 may be plastic or metallic. It is advantageously made of a high strength deformable material such as stainless steel. Alternatively, it may be made of an elastic material or superelastic material such as Nitinol. The device may be stretched over to exert radial force on the stent to help hold the stent in a collapsed configuration.

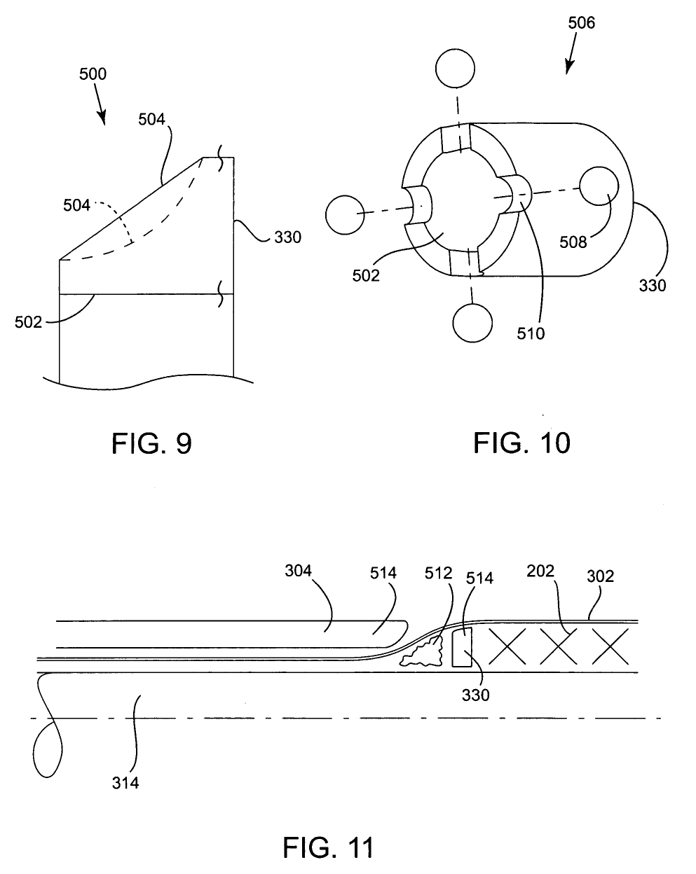

In FIG. 9 another type of stop member 500 is shown. It is of a type optionally employed in the variation of the invention pictured in FIGS. 6A and 6B (as are those in the remaining figures). Regarding stop 500, it is shown in partial side view. It includes a lumen 502 for receiving an inner member. Its length may be varied as indicated by the section breaks. The stop includes a stent abutment surface 330 and a bearing surface 504 for interfacing with a complementary surface of an end of the delivery device sleeve or an intermediate member. The bearing surface may be conical or concave or another shape to assist the restraint in making its diameter adaptive transition. In any case, the bearing system may be regarded as a bushing (non-rolling or planar-bearing) type approach.

The stop 506 variation in FIG. 10 includes a block and a plurality of rolling bearings 508. Four ball bearings are shown for the sake of convenience, but more are advantageously employed. These bearings may be captured in a cup or receptacle 510. Ball bearings are shown, but cylindrical bearings might alternatively be employed.

FIG. 11 shows a delivery system employing a fluid mass or bolus 512 to help transition the restraint down in size—thereby serving as a fluid bearing. The figure also shows a complementary end portion 514 of sleeve 304. Further, rather than relying on the stent to hold-in the fluid (which may be a medical-grade grease, for example) a blocker 516 may be provided. It will provided a seal for the bearing proximally and a stent stop surface distally. In order that the fluid bearing not leak out, the restraint is preferably imperforate and tolerances with the relevant members closely maintained. Additionaly, the bolus may be a high viscosity fluid, gel or sol that is essentially non-flowable.

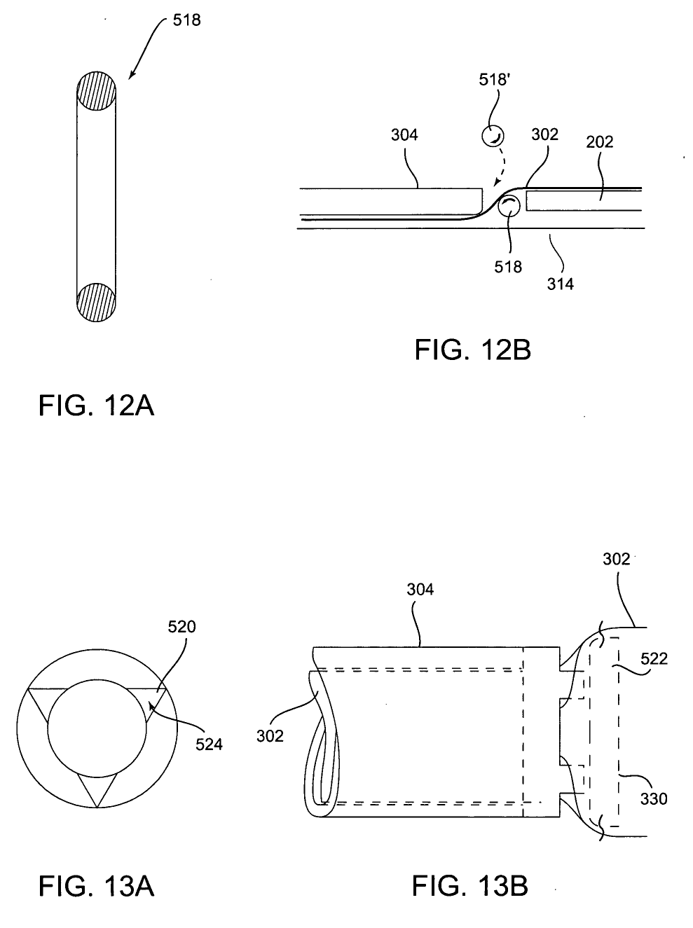

FIG. 12A is a side-sectional view of a stent stop in the form of a roller bearing. Essentially, the device may be a flexible standard O-ring 518. It may be comprise a flouropolymer, such as Viton® or silicone or possibly even be a Nitinol ring. As illustrated in FIG. 12B the bearing may be intended to roll as the restraint pulls past it. A second O-ring 518′ may be provided as a complimentary bearing surface with a placement as indicated as well.

FIG. 13A is an end view of a delivery device proximal to a stent including restraint cutter and/or stent stop features 520. As illustrated in the side view provided in FIG. 13B, these features may be directly provided in the sleeve or in a supplemental member (as indicated by the phantom line). Where no blocker 522 is provided the facing surface of each feature 520 will abut the stent. Otherwise blocker 522 will include the stent stop surface 330. FIG. 13B also shows the manner in which the restrain 302 can be received by such a system within pass-through openings 524 defined between adjacent ones of features 520.

In regard to any such system, it is to be understood that conventional materials and techniques may be employed in the system construction. In this regard, it will often be desired to provide a lubricious coating or cover between moving components to reduce internal system friction.

In addition, it is to be understood that various radiopaque markers or features may be employed in the system to 1) locate stent position and length, 2) indicated device actuation and stent delivery and/or 3) locate the distal end of the delivery guide. As such, various platinum (or other radiopaque material) bands or other markers (such as tantalum plugs) may be variously incorporated into the system. Alternatively, or additionally, the stent stop or blocker member may be made of radiopaque material. Especially where the stent employed may shorten somewhat upon deployment, it may also be desired to align radiopaque features with the expected location (relative to the body of the guide member) of the stent upon deployment. For example, it may be desired to incorporate radiopaque features into the restraint and/or bridge or connector sections so that the deployment motion of the device is visible under fluoroscopy. Exemplary markers that may be of use are shown at a proximal end of the stent in FIG. 4 as elements A and A′—on the delivery guide body and restraint, respectively—and at a distal end of the stent on the restraint as element B.

Though the invention has been described in reference to several examples, optionally incorporating various features, the invention is not to be limited to that which is described or indicated as contemplated with respect to each embodiment or variation of the invention. The breadth of the present invention is to be limited only by the literal or equitable scope of the following claims. That being said, we claim:

1. A stent delivery system comprising:
a stent; and
a delivery guide comprising a restraint covering the stent in a collapsed configuration, a length of the restraint having a first diameter over the stent, the length of the restraint adapted to assume a substantially reduced second diameter when withdrawn from the stent.

2. The system of claim 1, wherein the stent is a self-expanding stent and the restraint holds the stent in the collapsed configuration.

3. The system of claim 1, wherein the delivery guide further comprises an atraumatic distal tip.

4. The system of claim 1, wherein the delivery guide further comprises:
an inner member,
an outer sleeve, the inner member slidingly received by the sleeve; and
a stop surface to abut at least a portion of the stent,
wherein an outer diameter of the collapsed stent is greater than a distal inner diameter of the sleeve, and the inner diameter is greater than the restraint second diameter.

5. The system of claim 4, wherein the inner member is a corewire.

6. The system of claim 5, wherein the restraint fits within the sleeve by at least partially filling space occupied by the collapsed stent when the restraint is withdrawn.

7. The system of claim 5, wherein the restraint is connected to the corewire so a distal end of the core wire moves with the restraint.

8. The system of claim 7, further comprising an extension wire connected to the sleeve distal to the core wire.

9. The system of claim 8, further comprising a bridge segment connecting the restraint to the core member.

10. The system of claim 4, wherein a distal end of the system is adjustable relative to the sleeve.

11. The system of claim 10, further comprising a corewire with the distal tip.

12. The system of claim 11, wherein the inner member extends from the restraint.

13. The system of claim 1, wherein the second diameter is reduced from the first diameter by at least about 2%.

14. The system of claim 13, wherein the second diameter is reduced from the first diameter by up to about 50%

15. The system of claim 1, wherein the restraint is urged into said second diameter upon withdrawal.

16. The system of claim 1, wherein the first diameter is as large as about 0.028 inch.

17. The system of claim 17, wherein the first diameter is as large as about 0.018 inch

18. The system of claim 18, wherein the first diameter is as large as about 0.014 inch.

19. The system of claim 4, further comprising a stopper, the stopper providing the surface for abutting the portion of the stent.

20. The system of claim 19, wherein the stopper defines openings at a proximal end for receiving sections of the restraint.

21. The system of claim 20, wherein a wedge is provided between adjacent ones of the openings.

22. The system of claim 21, wherein a sharp edge is provided between adjacent ones of the openings.

23. The system of claim 20, wherein the restraint comprises separate sections.

24. The system of claim 20, wherein the restraint is adapted for separating into sections.

25. The system of claim 19, wherein the stopper is internal to the restraint.

26. The system of claim 19, wherein the stopper is external to the restraint.

27. The system of claim 19, wherein the stopper and a distal end of the sleeve provide a cooperative bearing surfaces to transition the restraint from the first diameter to the second diameter.

28. The system of claim 27, wherein the stopper comprises a plurality of bearings and a retainer ring distal to the ball bearings.

29. The system of claim 28, wherein the bearings are ball bearings.

30. The system of claim 27, wherein the stopper comprises a fluid bearing and a retainer ring distal to the fluid bearing.

31. The system of claim 30, wherein the fluid bearing comprises medical grease.

32. The system of claim 27, wherein the stopper comprises a flexible ring.

33. The system of claim 20, wherein the restraint comprises a collapsible wire-mesh member.

34. A method of stent delivery, the method comprising:
providing a stent delivery system having a restraint holding a self-expanding stent in a collapsed configuration,
positioning the stent at a target site, and
withdrawing the restraint to release the stent for deployment at the target site,
wherein at least a portion of the restraint substantially decreases in diameter during release of the stent.

35. The method of claim 34, wherein withdrawing in inner member of the delivery system withdraws the restraint.

36. A method of stent delivery, the method comprising:
providing a stent delivery system having a restraint holding a self-expanding stent in a collapsed configuration,
positioning the stent at a target site, and
withdrawing the restraint to release the stent for deployment at the target site,
wherein at least a portion of the restraint is urged into a decreased diameter during release of the stent.

37. The method of claim 34, wherein withdrawing in inner member of the delivery system withdraws the restraint.
